(19) United States
(12) Patent Application Publication
(10) Pub. No.: US 2013/0174299 A1
(43) Pub. Date: Jul. 4, 2013

(54) METHOD FOR PRODUCTION OF STOLON-FORMING PLANT HAVING IMPROVED TUBER PRODUCTION ABILITY OR STOLON PRODUCTION ABILITY COMPARED WITH WILD TYPE, AND STOLON-FORMING PLANT PRODUCED BY THE METHOD

(71) Applicants: Akiho Yokota, Ikoma-shi (JP); Hiroki Ashida, Ikoma-shi (JP); Kinya Akashi, Ikoma-shi (JP); Keiichi Ushiyama, Ikoma-shi (JP); Shigeru Shigeoka, Nara-shi (JP)

(72) Inventors: Akiho Yokota, Ikoma-shi (JP); Hiroki Ashida, Ikoma-shi (JP); Kinya Akashi, Ikoma-shi (JP); Keiichi Ushiyama, Ikoma-shi (JP); Shigeru Shigeoka, Nara-shi (JP)

(21) Appl. No.: 13/823,375

(22) PCT Filed: Apr. 11, 2011
(86) PCT No.: PCT/JP2011/059034

§ 371 Date: Mar. 14, 2013

(30) Foreign Application Priority Data
Sep. 22, 2010 (JP) 2010-211854

(51) Int. Cl. C12N 15/82

(52) U.S. Cl. CPC C12N 15/8201
USPC 800/290; 800/298; 536/23.6

(57) ABSTRACT

The present invention provides: a method for producing stolon-forming plant having an improved tuber production ability as compared with a corresponding wild strain; and a stolon-forming plant produced by the method. The present invention also provides: a method for producing a stolon-forming plant having an improved stolon formation ability as compared with a corresponding wild strain; a stolon-forming plant produced by the method; and a kit for improving a stolon formation ability of a stolon-forming plant. The method of the present invention is a method for producing a stolon-forming plant having an improved tuber production ability as compared with a corresponding wild strain, the method including the step of introducing, into a stolon-forming plant, a Ran gene derived from Citrullus lanatus. The method of the present invention can further include the step of introducing, into a stolon-forming plant, a polynucleotide encoding fructose-1,6-bisphosphatase/sedoheptulose-1,7-bisphosphatase derived from a blue-green alga.

### TECHNICAL FIELD

The present invention relates to a method for producing a stolon-forming plant having an improved tuber production ability as compared with a corresponding wild strain; and a stolon-forming plant produced by the method. The present invention also relates to a kit for improving a tuber production ability of a stolon-forming plant.

The present invention also encompasses a method for producing a stolon-forming plant having an improved stolon formation ability as compared with a corresponding wild strain; a stolon-forming plant produced by the method; a kit for improving a stolon formation ability of a stolon-forming plant.

### BACKGROUND ART

The year 2008 has been declared International Year of Potato, for potato plants by the United Nations. According to the United Nations, the world population is expected to increase by an average of hundred million or more per year within the next 20 years. Food shortage caused by the population increase has been a global issue. In particular, more than 95% of the population increase is considered to be taking place in developing countries. Tuberous plants including potato plants (Solanum tuberosum) are highly nutritious crops and meet cultivation conditions (e.g., cultivation in a small land is possible) required in developing countries. For this reason, activities for promoting potato plants are carried out widely. In India, research for doubling the amount of production of potatoes within five to ten years has been started. In south of Sahara in Africa, the amount of production of potatoes is increasing more rapidly than any other crops. If the amount of consumption of potatoes continues to increase in the future, farmers cultivating a large part of potato plants in the developing countries will have an increased income. Potato plants are therefore considered to be a crop which is beneficial for food security as well as creation of income.

However, in order to achieve these, it is necessary to increase a yield of a crop (e.g., tuberous plants such as potato plants) per unit area of farmland (i.e., increase a crop yield per individual plant), since the area of farmland is limited. As such, there has been conducted a great deal of research on improving a crop yield per individual plant by use of gene recombination technology.

For example, Non-patent Literature 1 discloses that a key enzyme SPS (sucrose-phosphate synthase) gene for synthesizing sucrose, which is a starting material for starch synthesis, was introduced into a potato plant to accelerate a metabolic function of starch, so that the productivity of tubers (edible parts) of the potato plant was improved by 1.2 times. Further, Non-patent Literature 2 discloses that an ADK (adenylate kinase) gene was introduced into a potato plant to accelerate a metabolic function of starch, so that the productivity of tubers (edible parts) of the potato plant was improved by about two times.

On the other hand, the inventors of the present invention introduced, to a tobacco plant, a gene encoding fructose-1,6-bisphosphatase/sedoheptulose-1,7-bisphosphatase derived from a blue-green alga, and caused the expression of the gene in a chloroplast of the tobacco plant. This allowed the inventors to successfully accelerate the growth of the plant by improving a photosynthetic function (see Patent Literature 1).

Further, the inventors of the present invention newly found, in Citrullus lanatus native to Botswana, a gene (Ran gene) having an activity of accelerating elongation of a root of a plant, and introduced the Ran gene into Arabidopsis thaliana. This allowed the inventors to successfully obtain Arabidopsis thaliana having an accelerated elongation of a root (see Patent Literature 2).

### CITATION LIST

### Patent Literature

Patent Literature 1
Japanese Patent Application Publication, Tokukai, No. 2002-300821 A (Publication Date: Oct. 15, 2002)
Patent Literature 2
International Publication No. 2007/100094, Pamphlet (Publication Date: Sep. 7, 2007)

### Non-Patent Literature

Non-patent Literature 1
Plant Prod. Sci. 2(2), 92-9 (1999)
Non-patent Literature 2
Nature Biotechnol. 20(12), 1256-60 (2002)

### SUMMARY OF INVENTION

### Technical Problem

As described in Background Art, in order to solve the issue of food shortage, there has been conducted research aimed at increasing the yield of a staple product eaten all around the world, tuberous plants, particularly potato plants. However, up to now, the yield of tubers (edible part) of a transgenic potato plant has been increased by use of gene recombination technology only to about double the yield of a corresponding wild strain.

The present invention is accomplished in view of the above problem. An object of the present invention is to provide means for producing a stolon-forming plant, such as a potato plant, which has a notably improved tuber production ability as compared with a corresponding wild strain; and a stolon-forming plant produced by the method.

Another object of the present invention is to provide a method for producing a stolon-forming plant having an improved stolon formation ability as compared with a corresponding wild strain; a stolon-forming plant produced by the method; and a kit for improving a stolon formation ability of a stolon-forming plant.

### Solution to Problem

After diligent study, the inventors of the present invention found, by chance, that introducing, into a potato plant, a gene (Ran gene) having an activity of promoting elongation of a root of a plant increases the weight and number of tubers. The inventors also found that introducing the Ran gene into a potato plant makes it possible to increase the number of stolons. After further study, the inventors found that introducing, into a potato plant, a gene (gene encoding fructose-1,6-bisphosphatase/sedoheptulose-1,7-bisphosphatase derived from a blue-green alga) having a function of accelerating photosynthesis in addition to the Ran gene increases an yield of tubers of the potato plant. Based on these finding, the inventors achieved the present invention. The increase in yield of the potato plant was far greater than an increase which had been reported in the past.

Note that the Ran gene is only known to have the function of accelerating elongation of a root, and a person skilled in the art cannot expect that the Ran gene contributes to improving an ability of producing a tuber, which is stem tissue, and a photosynthesis ability of the tuber.

A production method in accordance with the present invention is a method for producing a stolon-forming plant having an improved tuber production ability as compared with a corresponding wild strain, the method including the step of introducing, into a stolon-forming plant, a Ran gene derived from Citrullus lanatus.

A stolon-forming plant in accordance with the present invention is a stolon-forming plant having an improved tuber production ability as compared with a corresponding wild strain, the stolon-forming plant being produced by the production method.

A kit in accordance with the present invention is a kit for improving a tuber production ability of a stolon-forming plant, the kit including: a Ran gene derived from Citrullus lanatus; and a polynucleotide encoding fructose-1,6-bisphosphatase/sedoheptulose-1,7-bisphosphatase derived from a blue-green alga.

The present invention also encompasses a method for producing a stolon-forming plant having an improved stolon formation ability as compared with a corresponding wild strain, the method including the step of introducing, into a stolon-forming plant, a Ran gene derived from Citrullus lanatus.

The present invention also encompasses a stolon-forming plant having an improved stolon formation ability as compared with a corresponding wild strain, the stolon-forming plant being produced by the production method.

The present invention also encompasses a kit for improving a stolon formation ability of a stolon-forming plant, the kit including a Ran gene derived from Citrullus lanatus.

### Advantageous Effects of Invention

The present invention makes it possible to provide a method for producing stolon-forming plant having an improved tuber production ability as compared with a corresponding wild strain; and a stolon-forming plant produced by the method. The present invention also makes it possible to provide a kit for improving a tuber production ability of a stolon-forming plant.

Further, the present invention makes it possible to provide a method for producing a stolon-forming plant having an improved stolon formation ability as compared with a corresponding wild strain; a stolon-forming plant produced by the method; and a kit for improving a stolon formation ability of a stolon-forming plant.

Therefore, according to the present invention, it is possible to provide means for solving the global issue of food shortage.

### BRIEF DESCRIPTION OF DRAWINGS

FIG. 1 is a schematic view illustrating a mechanism of action of improving a tuber production ability in the present invention.

FIG. 2 is a schematic view illustrating a structure of a gene architecture introduced into a potato plant in examples and a comparative example.

FIG. 3 is agarose gel electrophoretograms of PCR products for checking, by PCR, whether or not genes were introduced into transgenic potato plants obtained in the examples and the comparative example.

FIG. 4 is a view showing results of checking, by western blotting, whether or not each of the genes introduced were expressed in each of the transgenic potato plants (fifth week of cultivation) obtained in the examples and the comparative example. (a) of FIG. 4 shows whether or not a Ran protein was detected, and (b) of FIG. 4 shows whether or not FBPase/SBPase was detected.

(a) of FIG. 5 is a photograph of entire plant bodies of the transgenic potato plants (fifth week of cultivation) obtained in the examples and the comparative example. (b) of FIG. 5 is a photograph of portions around rhizome parts and root parts of the transgenic potato plants (fifth week of cultivation). (c) of FIG. 5 is a photograph of tubers harvested from the transgenic potato plants (fifth week of cultivation).

(a) of FIG. 6 is a graph of comparison between the transgenic potato plants (fifth week of cultivation), obtained in the examples and the comparative example, in terms of height of an aerial part. (b) of FIG. 6 is a graph of comparison between the transgenic potato plants (fifth week of cultivation), obtained in the examples and the comparative example, in terms of dry weight of an aerial part. (c) of FIG. 6 is a graph of comparison between the transgenic potato plants (fifth week of cultivation), obtained in the examples and the comparative example, in terms of number of stolons (stolon count). (d) of FIG. 6 is a graph of comparison between the transgenic potato plants (fifth week of cultivation) in terms of number of tubers (tuber count). (e) of FIG. 6 is a graph of comparison between the transgenic potato plants (fifth week of cultivation) in terms of weight of tubers (tuber weight). (f) of FIG. 6 is a graph of comparison between the transgenic potato plants (fifth week of cultivation) in terms of photosynthesis rate.

(a) and (b) of FIG. 7 are photographs of the tubers of the transgenic potato plants obtained in the examples and the comparative example, the photograph of (a) being taken one month after the tubers were subjected to the germination ability test, the photograph of (b) being taken after two month after the tubers were subjected to the germination ability test.

### DESCRIPTION OF EMBODIMENTS

The following description will discuss an embodiment of the present invention in detail.

<1. Method for Producing Stolon-Forming Plant>

A method (referred to as “production method of the present invention”) for producing a stolon-forming plant in accordance with the present invention includes a step of introducing a Ran gene (referred to as “Ran gene introduction step”). The method for producing a stolon-forming plant can include, in addition to the Ran gene introduction step, a step (FBPase/SBPase gene introduction step) of introducing a polynucleotide (FBPase/SBPase gene) encoding fructose-1,6-bisphosphatase/sedoheptulose-1,7-bisphosphatase.

Another step that may be included in production of a stolon-forming plant can be further included as well as the above introduction steps. Examples of the another step encompass a step of culturing the stolon-forming plant after the introduction step or steps.

As used herein, the term “fructose-1,6-bisphosphatase/sedoheptulose-1,7-bisphosphatase” is synonymous with “fructose-1,6-/sedoheptulose-1,7-bisphosphatase.” As used herein, the term “FBPase/SBPase” is synonymous with “FBP/SBPase.”

Note that a tuber production ability can be improved better in a case where the Ran gene derived from Citrullus lanatus and the FBPase/SBPase gene derived from a blue-green alga are introduced together into a stolon-forming plant as compared with a case where the Ran gene derived from Citrullus lanatus and the FBPase/SBPase gene derived from a blue-green alga are each introduced alone into a stolon-forming plant.

<1-1. Ran Gene Introduction Step>

According to the Ran gene introduction step, a Ran gene is introduced into a stolon-forming plant, so that a stolon-forming plant having an improved tuber production ability as compared with a corresponding wild strain can be produced.

A production method in accordance with the present invention is a method for producing a stolon-forming plant having an improved tuber production ability as compared with a corresponding wild strain, the method including the step of introducing, into a stolon-forming plant, a Ran gene derived from Citrullus lanatus.

The production method in accordance with the present invention can be arranged such that the Ran gene is the following (A) or (B):

(A) a polynucleotide consisting of the base sequence of SEQ ID NO: 1 or 2; and

(B) a polynucleotide that is hybridizable, under a stringent condition, with a polynucleotide consisting of a complementary base sequence of the polynucleotide consisting of the base sequence of SEQ ID NO: 1 or 2 and encodes a polypeptide having an activity of improving a tuber production ability.

The production method in accordance with the present invention can be arranged such that the Ran gene is a polynucleotide encoding a polypeptide of the following (C) or (D):

(C) a polypeptide consisting of the amino acid sequence of SEQ ID NO: 3 or 4; and

(D) a polypeptide (i) consisting of an amino acid sequence in which one or several amino acids are substituted, deleted, or added in the polypeptide consisting of the amino acid sequence of SEQ ID NO: 3 or 4 and (ii) having an activity of improving a tuber production ability.

Note that “a polypeptide having an activity of improving a tuber production ability” denotes a polypeptide which has such an activity that by introducing a polynucleotide encoding the polypeptide into a stolon-forming plant and causing the polynucleotide to be expressed, it is possible to produce a stolon-forming plant having an improved tuber production ability as compared with a corresponding wild strain.

Note that the “Ran gene” is derived from wild watermelon (Citrullus lanatus) which grows in the Kalahari Desert located in the area around the Republic of Botswana, Africa. A Ran gene suitably applicable to the production method of the present invention can be obtained by RT-PCR by use of (i), as a template, all mRNA which is prepared, by a method known to a person skilled in the art, from tissue derived from Citrullus lanatus, and (ii) a primer designed to amplify an entire length of the Ran gene. The Ran gene can also be obtained by PCR by use of (i), as a template, a cDNA library derived from Citrullus lanatus and (ii) a primer designed to amplify the entire length of the Ran gene.

Amplification of the Ran gene by PCR is carried out by repeating a cycle of raising and lowering a temperature in a reaction solution containing (i) a polynucleotide which serves as a template, (ii) a PCR buffer, (iii) a primer set (a forward primer and a reverse primer), (iv) a dNTP mixture (a mixture of deoxynucleotide triphosphate), and (v) a DNA polymerase.

In one embodiment of the present invention, the Ran gene may include a site substituted with a synthesized nucleotide. A site at which the Ran gene is substituted is not limited to a specific one, as long as a protein expressed from a base sequence obtained by the substitution has a suitable property.

The Ran gene can consist solely of a polynucleotide encoding a Ran protein derived from Citrullus lanatus, or can consist of the polynucleotide and another polynucleotide further added. Examples of a polynucleotide that can be added encompass, but not limited to, a polynucleotide (e.g., a histidine tag, a Myc tag, a FLAG tag, and the like) encoding a label protein; a polynucleotide encoding a protein, such as GST or MBP, which is to be fused with a Ran protein; a polynucleotide (e.g., a promoter sequence derived from yeast, a promoter sequence derived from a phage, a promoter sequence derived from E. coli, and the like) encoding a promoter sequence, and a polynucleotide encoding a signal sequence (e.g., an endoplasmic reticulum transport signal sequence, a secretory sequence, and the like). Parts to which these polynucleotides are added are not limited to specific ones, and can be such a part of a polynucleotide that is to be expressed at an N-terminal or a C-terminal of a protein to be translated from the polynucleotide.

The Ran gene encompasses (i) a polynucleotide (e.g., a polynucleotide having the base sequence of SEQ ID NO: 1 or 2) encoding a Ran protein derived from Citrullus lanatus or (ii) a polynucleotide that is hybridizable, under a stringent condition, with a polynucleotide consisting of a complementary base sequence of the polynucleotide encoding the Ran protein derived from Citrullus lanatus. That is, the Ran gene is not limited to the polynucleotide having the base sequence of SEQ ID NO: 1 or 2, as long as the Ran gene functions as a Ran gene of the present invention. Accordingly, the Ran gene encompasses a mutant of the Ran gene. Such a polynucleotide is considered to encode a polypeptide having an activity of improving a tuber production ability, and therefore provides an effect desired in the present invention.

As used herein, the term “stringent condition” means a condition that hybridization is carried out at a temperature ranging from a Tₘ value of nucleic acids which are highly homologous with each other (e.g., a Tₘ value of a completely matching hybrid) to a temperature lower than the Tₘ value by 15° C., preferably by 10° C. Specifically, the term “stringent condition” means a condition that hybridization is carried out in a generally-used buffer solution for hybridization at 68° C. for 20 hours. In other words, the “stringent condition” means a condition that hybridization is caused only in a case where there is an identity of at least 80% or more, preferably an identity of at least 90% or more, more preferably an identity of at least 95% or more, and most preferably an identity of 97% or more between sequences.

A base sequence of a Ran gene used in the production method of the present invention can be determined by the dideoxy chain termination method described in a reference: Science., 214(4526), 1205-10 (1981).

A recombinant vector suitably applicable to the production method of the present invention can be obtained by inserting the Ran gene into a vector for plant transformation. A vector for plant cell transformation is not limited to a specific one, but, for example, (i) pBI101, pBI121, and pBI122 (manufactured by Clontech), PBE2113-GUS (the National Institute of Agrobiological Sciences), and the like which are derived from a Ti plasmid and (ii) a Geminivirus vector (such as WDV), a vector (such as a virus vector derived from a tobacco mosaic virus, a cucumber mosaic virus, or a potyvirus) derived from an RNA virus, and the like. Further, it is possible to (i) introduce, directly into a plant cell, a polynucleotide fragment of a Ran gene cut out from a vector, or (ii) integrate, directly into a plant genome, a polynucleotide fragment that lacks a promoter and/or a polyA and regulate transcription by means of an endogenous promoter and/or a polyA signal.

A recombinant vector suitably applicable to the present invention can be constructed by ligation of (i) a fragment of the polynucleotide which is cut out, by restriction enzyme treatment or the like, from the Ran gene which has been separated and purified and (ii) a linear polynucleotide obtained by cutting, by use of a restriction enzyme, a vector which serves as a base. For the ligation, a DNA ligase or the like can be used in accordance with properties of the vector and the polynucleotide. It is possible to obtain a transformant of a plant containing the Ran gene by introducing the recombinant vector into a replicable host and then carrying out screening by use of, as indicators, (i) a marker of the recombinant vector and (ii) expression of enzyme activity. It is therefore preferable that the vector contain a marker gene such as a drug resistance gene.

Note that a vector which is constructed by use of a polypeptide having the base sequence of SEQ ID NO: 1 and a vector which is constructed by use of a polypeptide having the base sequence of SEQ ID NO: 2 can be used as an expression vector for causing expression of a polypeptide having the amino acid sequence of SEQ ID NO: 3 and an expression vector for causing expression of a polypeptide having the amino acid sequence of SEQ ID NO: 4, respectively.

According to the production method of the present invention, the recombinant vector is introduced into a target host plant, so that a plant having an improved tuber productivity can be produced.

A plant on which the production method of the present invention has an effect of accelerating tuber production ability is a plant that can produce a tuber. A tuber is an enlarged end section of a stolon where starch and other nutrient are accumulated. It follows that the plant on which the production method of the present invention has the effect of accelerating tuber production ability is a plant (referred to as “stolon-forming plant”) that can form a stolon. A stolon-forming plant to which the production method of the present invention is applied is not limited to a specific one, and can be, for example, potato, arrowhead, taro, konjac, lotus root, Jerusalem artichoke, ginger, licorice, and the like.

As shown with the examples described below, introduction of a Ran gene into a stolon-forming plant increases the number of stolons as compared with a corresponding wild strain. It follows that the present invention encompasses a method for producing a stolon-forming plant having an improved stolon formation ability as compared with a corresponding wild strain, the method including a step of introducing the Ran gene into a stolon-forming plant. In this case, a plant to which the present invention is applied can be, in addition to the stolon-forming plant which produces a tuber, a stolon-forming plant (e.g., strawberry, strawberry saxifrage, or the like) which does not produce a tuber. A strawberry plant or the like forms a stolon above ground, and an edible part (fruit) is formed on the stolon. Accordingly, improvement of stolon formation ability (i.e., increase in number of stolons) may be able to increase an yield of edible parts (fruits) of the strawberry plant. Note that a description of the method, of the present invention, for producing a stolon-forming plant having an improved tuber production ability as compared with a corresponding wild strain can also serve as a description of the method, of the present invention, for producing a stolon-forming plant having an improved stolon formation ability as compared with a corresponding wild strain.

A form of a host plant and a part of the host plant where introduction is carried out can be plant cultured cell; callus; protoplast; an entire plant body of a cultivated plant: plant organs such as leaf, petal, stalk, root, rhizome, and seed; and plant tissue such as epidermis, phloem, parenchyma, xylem, and vascular bundle.

A method for introducing a Ran gene into a host plant can be a method known to a person skilled in the art, such as a method in which bacteria of the genus Agrobacterium is used (the Agrobacterium method), electroporation, biolistic transformation, and microinjection. The Agrobacterium method, which is suitable as a method for plant transformation, is particularly preferable.

The Agrobacterium method is a well-known method and carried out, for example, by causing Agrobacterium bacteria, into which the recombinant vector has been introduced, to infect a cell or a section of a plant (see, e.g., reference: Proc. Natl. Acad. Sci. USA., 94(6), 2117-21 (1997)).

In the present invention, all of the Ran genes derived from Citrullus lanatus as described in Patent Literature 2 and all of the proteins transcribed and translated from the Ran genes can be suitably used. Even in a case where deletion, substitution, or addition is carried out in an amino acid sequence of the Ran protein, the Ran protein is within the scope of the present invention as long as the Ran protein maintains a function as a Ran protein.

<1-2. FBPase/SBPase Gene Introduction Step>

The production method in accordance with the present invention can further include the step of introducing, into the stolon-forming plant, a polynucleotide encoding FBPase/SBPase derived from a blue-green alga.

The production method in accordance with the present invention can be arranged such that the polynucleotide encoding FBPase/SBPase derived from a blue-green alga is the following (E) or (F):

(E) a polynucleotide consisting of the base sequence of SEQ ID NO: 5; and

(F) a polynucleotide that is hybridizable, under a stringent condition, with a polynucleotide consisting of a complementary base sequence of the polynucleotide (E) and encodes a polypeptide having FBPase/SBPase activity.

The production method in accordance with the present invention can be arranged such that the polypeptide encoding FBPase/SBPase derived from a blue-green alga is the following (G) or (H):

(G) a polypeptide consisting of the amino acid sequence of SEQ ID NO: 6; and

(H) a polypeptide consisting of an amino acid sequence in which one or several amino acids are substituted, deleted, or added in the polypeptide (G) and having FBPase/SBPase activity.

Note that FBPase/SBPase derived from a blue-green alga (Synechococcus PCC 7942) is a gene encoding an enzyme that is widely distributed in a blue-green alga, which belongs to prokaryotic algae. Also note that a protein transcribed and translated from the gene is a bifunctional enzyme which by itself has two enzyme activities: FBPase activity and SBPase activity. FBPase/SBPase is described in a reference “Archives of Biotechnology and Biophysics, Vol. 334, No 1, pp. 27 to 36, 1996 ‘Molecular characterization and resistance to hydrogen peroxide of two fructose-1,6-bisphosphatase from Synechococcus PCC 7942’”. “FBPase/SBPase gene” is registered in Genbank with Accession No. D83512.

In the present invention, all of the FBPase/SBPase genes derived from a blue-green alga as disclosed in Patent Literature 1 and all of the proteins transcribed and translated from the FBPase/SBPase genes can be suitably used. That is, even in a case where deletion, substitution, or addition is carried out in an amino acid sequence of the FBPase/SBPase protein, the FBPase/SBPase protein can be suitably applied to the production method of the present invention as long as the FBPase/SBPase protein maintains an enzymatic characteristic as an FBPase/SBPase protein.

In the FBPase/SBPase gene introduction step, a gene introduction method of the Ran gene introduction step can be suitably used. The Ran gene and the FBPase/SBPase gene can be introduced into a stolon-forming plant by use of respective different recombinant vectors, or can be introduced into the stolon-forming plant by use of a single recombinant vector, into which the Ran gene and the FBPase/SBPase gene have been inserted. The latter is more preferable in view of ease of recombinant operation and high gene introduction efficiency.

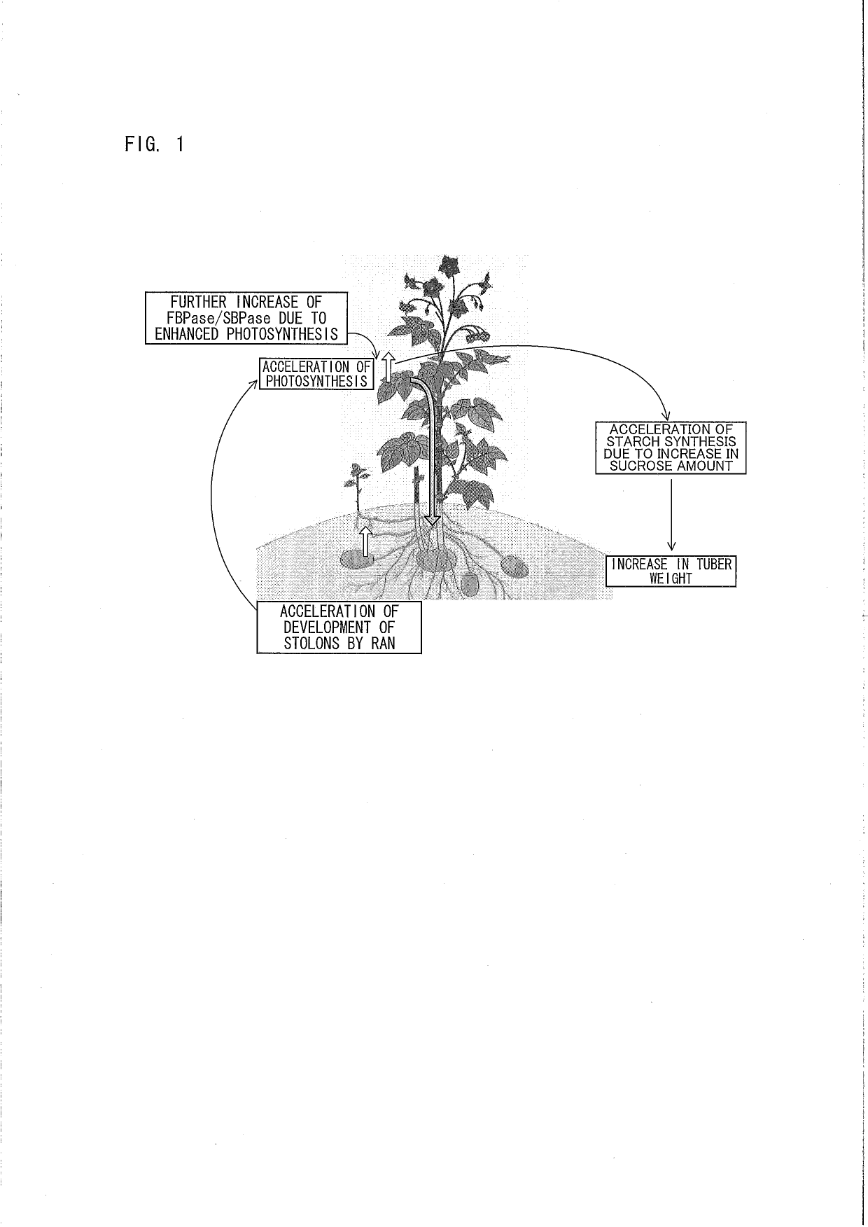

FIG. 1 shows a mechanism of action of the present invention. A Ran gene is introduced into a stolon-forming plant, so that development of a stolon is accelerated. This increases the number of parts to be tubers in the future. The increase in number of parts to be tubers causes an acceleration signal for starch synthesis to function, so that photosynthesis in a leaf in an aerial part is accelerated. This is considered to yield the following outcome. The activated photosynthesis increases an amount of sucrose synthesis as compared with a corresponding wild strain, so that sucrose (starting material for starch synthesis) is transferred to an underground part, where sucrose is converted into starch and accumulated in the form of starch in the stolon. This increases the weight and/or number of tubers as compared with a corresponding wild strain. Further, introducing the FBPase/SBPase gene in addition to the Ran gene accelerates the photosynthesis further as compared with a case where the Ran gene is introduced alone. This increases sucrose which serves as a starting material of starch, so that synthesis of starch is further accelerated. Consequently, tuber productivity is further improved.

As used herein, the wording “one or several amino acids are substituted, deleted, or added” means that a certain number of amino acids are substituted, deleted, or added, which number allows the amino acids to be substituted, deleted, or added by a publically-known mutant polypeptide production method such as site-directed mutagenesis (the range of the number is not limited to a specific one, but can be, for example, 1 to 40, preferably 1 to 20, more preferably 1 to 12, further more preferably 1 to 9, and particularly preferably 1 to 5). Such a mutant polypeptide is not limited to a polypeptide having a mutant which is introduced artificially by a publicly-known mutant polypeptide production method, and can be a polypeptide which is obtained by isolating and purifying a naturally-occurring polypeptide.

<2. Stolon-Forming Plant of Present Invention>

A stolon-forming plant in accordance with the present invention is a stolon-forming plant having an improved tuber production ability as compared with a corresponding wild strain, the stolon-forming plant being produced by the production method of the present invention. Note that “having an improved tuber production ability as compared with a corresponding wild strain” means that a transformed plant obtained by the production method of the present invention has increases in weight and/or number of tubers as compared with a stolon-forming plant (corresponding wild strain) on which the production method of the present invention has not been practiced. Whether or not the transformed plant has an improved tuber production ability as compared with a corresponding wild strain can be determined by studying a plurality of wild strains and a plurality of transformed plants in terms of weight and/or number of tubers. In a case where comparison of (i) an average weight and/or average number of tubers per individual of the plurality of wild strains and (ii) an average weight and/or average number of tubers per individual of the plurality of transformed plants indicates that the latter is greater than the former, it can be determined that the transformed plant has an improved tuber production ability as compared with the corresponding wild strain.

The stolon-forming plant having an improved tuber production ability as compared with the corresponding wild strain can be obtained by, after introducing the Ran gene into a host plant, selecting a plant cell into which the Ran gene has been introduced, causing callus tissue to be formed from the plant cell, and when a shoot appears from the callus tissue, transplanting the callus tissue to a rooting medium.

In order to select the plant cell into which the Ran gene has been introduced and cause the plant cell to form the callus tissue from which the shoot appears, it is preferable to employ, for example, the following culture condition. That is, a plant cell or a segment into which the Ran gene has been introduced is cultured, in the presence of a suitable agent for selection in accordance with a type of an introduced selection marker gene, on an agar medium such as Murashige-Skoog medium containing an appropriate amount of sucrose under a temperature condition of about 4° C. to 50° C., preferably about 15° C. to 37° C., more preferably about 22° C. to 30° C. for about 3 days to 180 days, preferably about 7 days to 90 days, and more preferably about 14 days to 60 days. An optimum temperature for culture can be specified with respect to each plant by culture experiment. Some types of a plant may require culturing for a longer period of time than the days presented above.

A shoot thus obtained is implanted to a publicly-known rooting medium such as Murashige-Skoog medium including 4 μM indolebutyric acid, and is cultured, for example, for about 3 days to 180 days, preferably about 7 days to 90 days, more preferably about 14 days to 60 days, so that the shoot becomes a plant body. If necessary, the plant body thus obtained can be transplanted into vermiculite or soil and cultivated.

Whether or not the Ran gene is incorporated in (i) a transgenic plant into which the Ran gene has been introduced and (ii) a next generation of the transgenic plant can be checked by detecting, by means of PCR, Southern analysis, or the like, the introduced Ran gene in genomic DNA of a cell or tissue of the transgenic plant and the next generation. For a case where the FBPase/SBPase gene is introduced in addition to the Ran gene, the above description should be read, replacing the terms “Ran gene” in the description above with “the Ran gene and the FBPase/SBPase gene” so as to be read as a description for a plant body into which both the Ran gene and the FBPase/SBPase gene have been introduced.

In one embodiment of the present invention, a plant body in which the Ran gene has been introduced alone has increases in weight and number of tubers per individual plant as compared with a corresponding wild strain. In one embodiment of the present invention, a plant body in which the Ran gene and the FBPase/SBPase gene have been introduced together has specific increases in weight and number of tubers per individual plant in spite of having little change in height and dry weight of an aerial part as compared with a corresponding wild strain. Since introduction of the FBPase/SBPase gene is known to accelerate growth of a plant and increase a size (including a height and dry weight of an aerial part) of the plant itself as compared with a corresponding wild strain (see Patent Literature 1), the plant body in which the Ran gene and the FBPase/SBPase gene had been introduced together was expected to have increases in height and dry weight of the aerial part as compared with a corresponding wild strain. However, unexpectedly, the plant body had no change in height of the aerial part but had the specific changes in weight and number of tubers per individual plant. This can be considered an unexpected effect in an arrangement in which the Ran gene and the FBPase/SBPase gene are introduced together.

The present invention encompasses a stolon-forming plant having an improved stolon formation ability as compared with a corresponding wild strain. Note that “having an improved stolon formation ability as compared with a corresponding wild strain” means that the number of stolons of a transformed plant obtained by the production method of the present invention is increased as compared with a stolon-forming plant (corresponding wild strain) on which the production method of the present invention is not practiced. Whether or not the transformed plant has an improved stolon formation ability as compared with a corresponding wild strain can be determined by studying a plurality of wild strains and a plurality of transformed plants in terms of number of stolons. In a case where comparison of (i) an average number of stolons per individual of the plurality of wild strains and (ii) an average number of stolons per individual of the plurality of transformed plants indicates that the latter is more than the former, it can be determined that the transformed plant has an improved stolon formation ability as compared with the corresponding wild strain.

<3. Kit of Present Invention>

A kit in accordance with the present invention is a kit for improving a tuber production ability of astolon-forming plant, the kit including: a Ran gene derived from Citrullus lanatus; and a polynucleotide encoding FBPase/SBPase derived from a blue-green alga.

The Ran gene derived from Citrullus lanatus, and the polynucleotide encoding FBPase/SBPase derived from a blue-green alga, both of which are contained in the kit of the present invention, are not limited to specific forms, and can be provided, for example, as an aqueous solution, a suspension, or freeze-dried powder. The freeze-dried powder can be produced by the law of the art.

Each of the Ran gene derived from Citrullus lanatus and the polynucleotide encoding FBPase/SBPase derived from a blue-green alga can contain an additive such as an excipient such as dextrin. A method for blending the additive is not limited to a specific one, and can be, for example, (i) a method of blending an additive agent with a buffer solution containing a Ran gene derived from Citrullus lanatus and a polynucleotide encoding FBPase/SBPase derived from a blue-green alga, (ii) a method of blending, with a buffer solution containing an additive agent, a Ran gene derived from Citrullus lanatus and a polynucleotide encoding FBPase/SBPase derived from a blue-green alga, (iii) a method of blending, with a buffer solution, a Ran gene, a polynucleotide encoding FBPase/SBPase derived from a blue-green alga, and a stabilizer at the same time, or (iv) the like.

The present invention also encompasses a kit for improving a stolon formation ability of a stolon-forming plant.

### CONCLUSION

The present invention encompasses the following invention.

A production method in accordance with the present invention is a method for producing a stolon-forming plant having an improved tuber production ability as compared with a corresponding wild strain, the method including the step of introducing, into a stolon-forming plant, a Ran gene derived from Citrullus lanatus.

The production method in accordance with the present invention can be arranged such that the Ran gene is the following (A) or (B):

(A) a polynucleotide consisting of the base sequence of SEQ ID NO: 1 or 2; and

(B) a polynucleotide that is hybridizable, under a stringent condition, with a polynucleotide consisting of a complementary base sequence of the polynucleotide consisting of the base sequence of SEQ ID NO: 1 or 2 and encodes a polypeptide having an activity of improving a tuber production ability.

The production method in accordance with the present invention can be arranged such that the Ran gene is a polynucleotide encoding a polypeptide of the following (C) of (D):

(C) a polypeptide consisting of the amino acid sequence of SEQ ID NO: 3 or 4; and

(D) a polypeptide (i) consisting of an amino acid sequence in which one or several amino acids are substituted, deleted, or added in the polypeptide consisting of the amino acid sequence of SEQ ID NO: 3 or 4 and (ii) having an activity of improving a tuber production ability.

The production method in accordance with the present invention can further include the step of introducing, into the stolon-forming plant, a polynucleotide encoding fructose-1,6-bisphosphatase/sedoheptulose-1,7-bisphosphatase derived from a blue-green alga.

The production method in accordance with the present invention is arranged such that the polynucleotide encoding fructose-1,6-bisphosphatase/sedoheptulose-1,7-bisphosphatase derived from a blue-green alga is the following (E) or (F):

(E) a polynucleotide consisting of the base sequence of SEQ ID NO: 5; and

(F) a polynucleotide that is hybridizable, under a stringent condition, with a polynucleotide consisting of a complementary base sequence of the polynucleotide consisting of the base sequence of SEQ ID NO: 5 and encodes a polypeptide having fructose-1,6-bisphosphatase/sedoheptulose-1,7-bisphosphatase activity.

The production method in accordance with the present invention is arranged such that the polynucleotide encoding fructose-1,6-bisphosphatase/sedoheptulose-1,7-bisphosphatase derived from a blue-green alga is a polynucleotide encoding a polypeptide of the following (G) or (H):

(G) a polypeptide consisting of the amino acid sequence of SEQ ID NO: 6; and

(H) a polypeptide (i) consisting of an amino acid sequence in which one or several amino acids are substituted, deleted, or added in the polypeptide consisting of the amino acid sequence of SEQ ID NO: 6 and (ii) having fructose-1,6-bisphosphatase/sedoheptulose-1,7-bisphosphatase activity.

A stolon-forming plant in accordance with the present invention is a stolon-forming plant having an improved tuber production ability as compared with a corresponding wild strain, the stolon-forming plant being produced by the production method.

A kit in accordance with the present invention is a kit for improving a tuber production ability of a stolon-forming plant, the kit comprising: a Ran gene derived from Citrullus lanatus; and a polynucleotide encoding fructose-1,6-bisphosphatase/sedoheptulose-1,7-bisphosphatase derived from a blue-green alga.

The present invention also encompasses a method for producing a stolon-forming plant having an improved stolon formation ability as compared with a corresponding wild strain, the method comprising the step of introducing, into a stolon-forming plant, a Ran gene derived from Citrullus lanatus.

At this time, the Ran gene can be the following (A) or (B):

(A) a polynucleotide consisting of the base sequence of SEQ ID NO: 1 or 2; and

(B) a polynucleotide that is hybridizable, under a stringent condition, with a polynucleotide consisting of a complementary base sequence of the polynucleotide consisting of the base sequence of SEQ ID NO: 1 or 2 and encodes a polypeptide having an activity of improving a stolon formation ability.

The Ran gene can also be a polynucleotide encoding a polypeptide of the following (C) or (D):

(C) a polypeptide consisting of the amino acid sequence of SEQ ID NO: 3 or 4; and

(D) a polypeptide (i) consisting of an amino acid sequence in which one or several amino acids are substituted, deleted, or added in the polypeptide consisting of the amino acid sequence of SEQ ID NO: 3 or 4 and (ii) having an activity of improving a stolon formation ability.

Further, the present invention also encompasses a stolon-forming plant having an improved stolon formation ability as compared with a corresponding wild strain, the stolon-forming plant being produced by the production method.

Further, the present invention also encompasses a kit for improving a stolon formation ability of a stolon-forming plant, the kit including a Ran gene derived from Citrullus lanatus.

The present invention is not limited to the above-described embodiments but allows various modifications within the scope of the claims. Any embodiment obtained by appropriately combining the technical means disclosed in the different embodiments will also be included in the technical scope of the present invention.

### EXAMPLES

The following description will discuss the present invention on the basis of examples.

[1. Construction of Transformation Vectors]

<1-1. Construction of FS Transformation Vector>

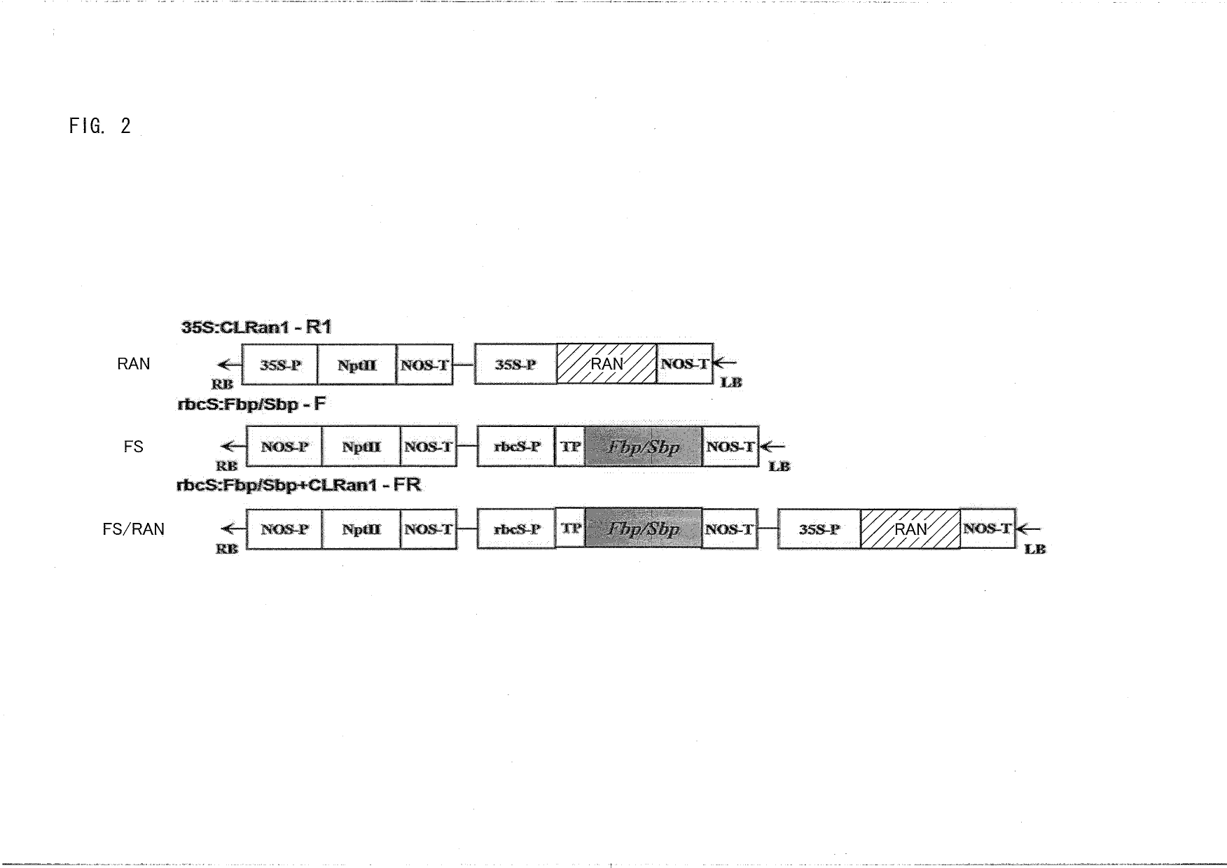

A DNA fragment obtained by connecting (i) a tomato rbcS (Ribulose bisphosphate carboxylase/oxygenase small subunit gene) promoter, to the 5′-terminal and 3′-terminal of which a HindIII restriction enzyme site and a SphI restriction enzyme site were added, respectively, (ii) a coding region of a transit peptide, to the 5′-terminal and 3′-terminal of which a HindIII restriction enzyme site and a SphI restriction enzyme site were added, respectively, and (iii) a FBPase/SBPase gene which was derived from a blue-green alga Synechococcus PCC7942 and to the 5′-terminal and 3′-terminal of which a SphI restriction enzyme site and a SacI restriction enzyme site were added, respectively, was inserted to a HindIII restriction enzyme site and a Sad restriction enzyme site of a pBI101 vector (Clontech) so that a β-glucuronidase gene of the pBI101 vector was replaced by the DNA fragment. In this way, a FS transformation vector (indicated as “FS” in FIG. 2) was prepared.

<1-2. Construction of RAN Transformation Vector>

Next, from full-length cDNA of RAN of Citrullus lanatus, a full length ORF was amplified by PCR by use of

(SEQ ID NO: 7)
5′-aaaaagcaggctccttttccaatggctt-3′
and
(SEQ ID NO: 8)
5′-agaaagctgggttttttactcgaacgcg-3′.

A PCR product thus obtained was introduced, through a recombination reaction by use of a GATEWAY system (Invitrogen), between a cauliflower mosaic virus 35S promoter and a nopaline synthase (NPTII) terminator of a vector pGWB2 for plant cell transformation. Thus prepared was a RAN transformation vector, into which a RAN gene was introduced (indicated as “RAN” in FIG. 2).

<1-3. Construction of FS/RAN Transformation Vector>

Next, by use of 5′-gaattcgcatgcctgcaggt-3′ (SEQ ID NO: 9) and 5′-gaattcccgatctagtaaca-3′ (SEQ ID NO: 10), in each of which an EcoRI site had been introduced, PCR was carried out based on the RAN transformation vector as a template, so that a DNA fragment in a region which ranged from the 35S promoter to the NPTII terminator and included the RAN gene was amplified (“gaattc” in the base sequence is an EcoRI part).

A PCR product thus obtained was treated with EcoRI and inserted to an EcoRI site of the FS vector. A plasmid made up of the 35S promoter, the RAN gene, and the NPTII terminator, which had been inserted in this direction downstream of an NPTII terminator for an FBPase/SBPase gene, was selected as an FS/RAN transformation vector (indicated as “FS/RAN” in FIG. 2).

[2. Transformation]

Each of the pBI121 (control vector), the FS transformation vector, the RAN transformation vector, and the FS/RAN transformation vector was introduced to an Agrobacterium (Agrobacterium tumefaciens) EHA105 strain by electroporation so as to be transformed.

A plurality of hypocotyls, each having a length of about 1 cm, were prepared from a potato plant (May Queen) cultured under aseptic conditions in a Murashige-Skoog (MS) agar medium, to which 3% sucrose had been added. Incubation was carried out at room temperature for 15 minutes by use of (i) the plurality of hypocotyls and (ii) Agrobacterium culture solutions containing the respective transformation vectors.

Subsequently, each of the hypocotyls was placed on a MS agar medium containing 0.4 mg/l indoleacetic acid and mg/l benzyladenine and was incubated at room temperature for two days.

Next, each of the hypocotyls was grown on a MS agar medium containing 2 mg/l benzyladenine, 0.1 mg/l gibberellic acid, 100 mg/l kanamycin, and 50 mg/l cefotaxime, so as to form a shoot. The shoot thus formed was transferred to a MS agar medium containing kanamycin and 50 mg/l cefotaxime, so as to root, so that a plant body was regenerated. The plant body regenerated was taken out of soil, and tubers were collected and preserved in a dark place at 4° C.

[3. Analysis of Transformants]

A tuber of a control potato plant, into which a gene had been introduced by use of the pBI121 (control vector), a tuber of a RAN transgenic potato plant, into which a gene had been introduced by use of the RAN transformation vector, a tuber of an FS transgenic potato plant, into which a gene had been introduced by use of the FS transformation vector, and a tuber of an FS/RAN transgenic potato plant, into which a gene had been introduced by use of the FS/RAN transformation vector, were each sprouted at room temperature. Then, each of the tubers was transplanted to a 3-lit. pot, which was filled with soil, and was grown for five weeks under the condition of 16-hour light/8-hour dark cycle with a temperature maintained at 18° C.

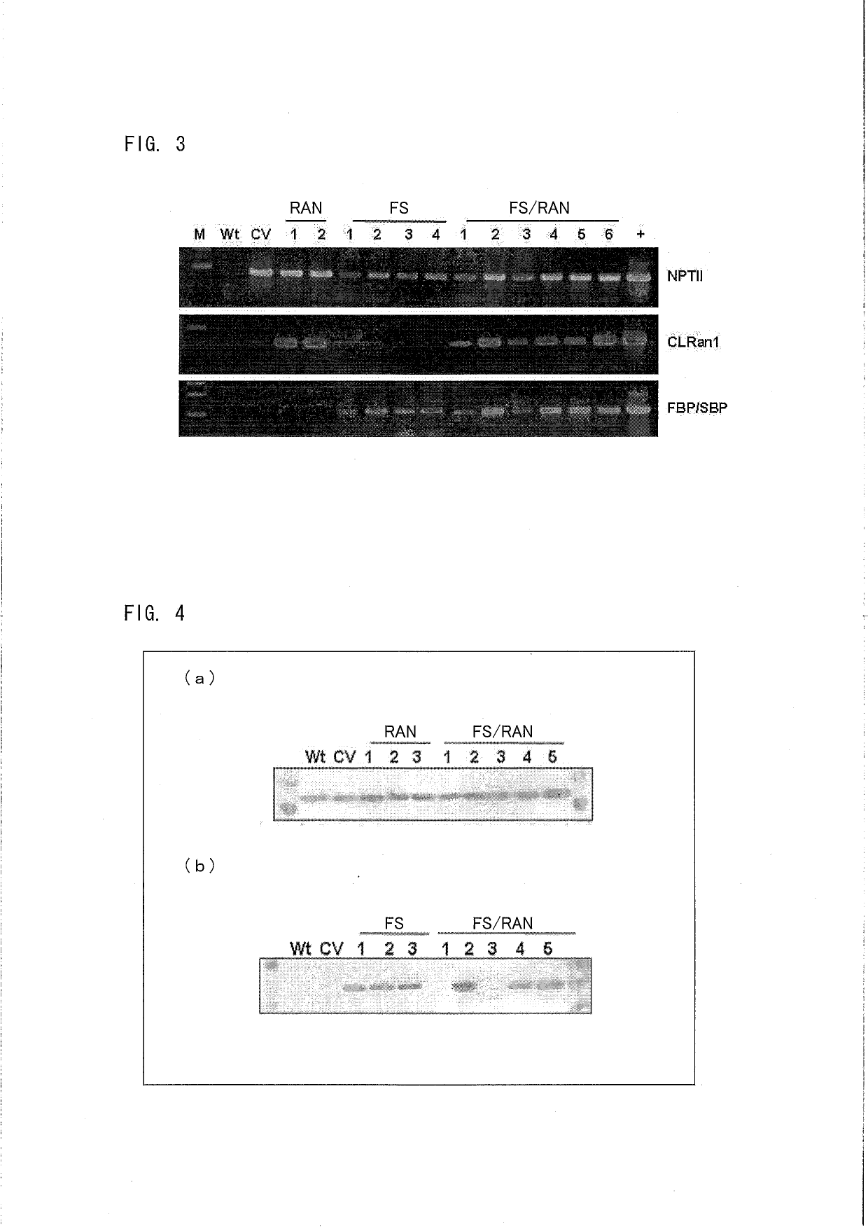

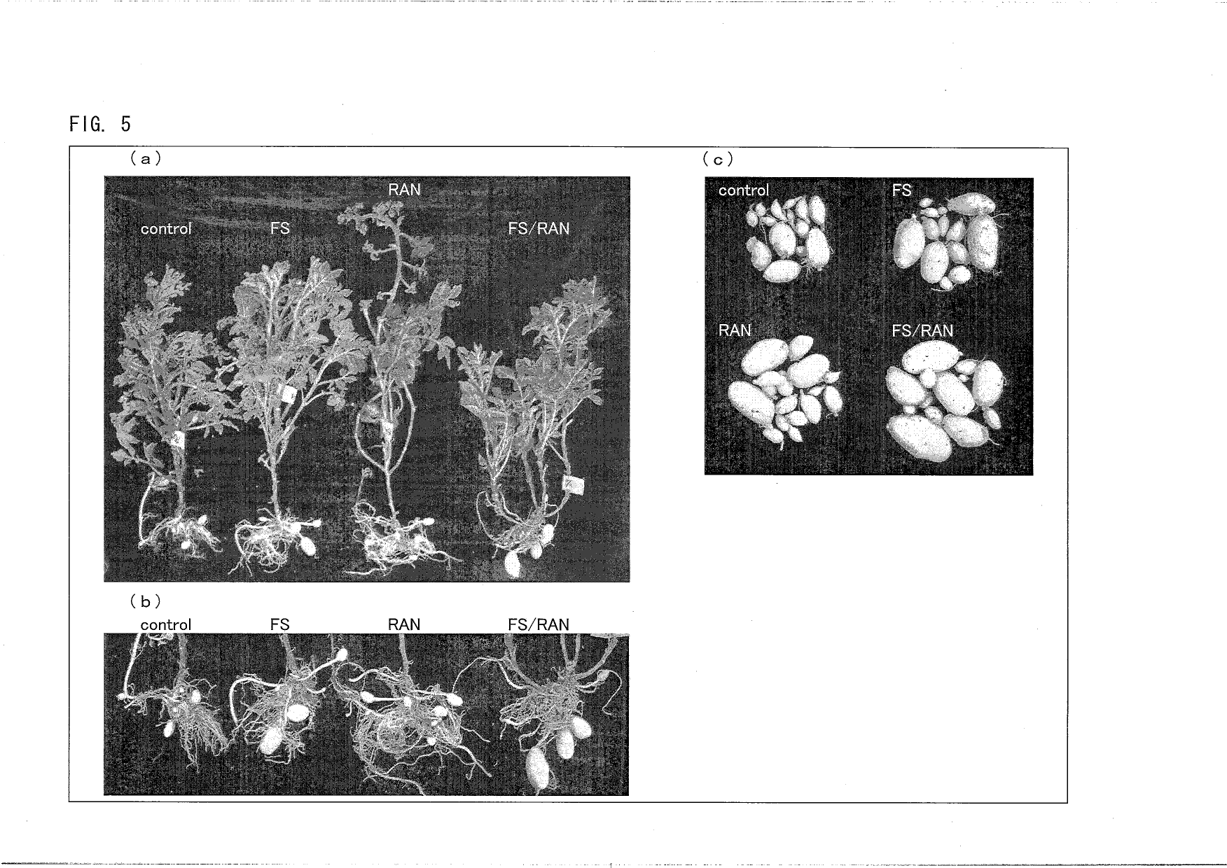

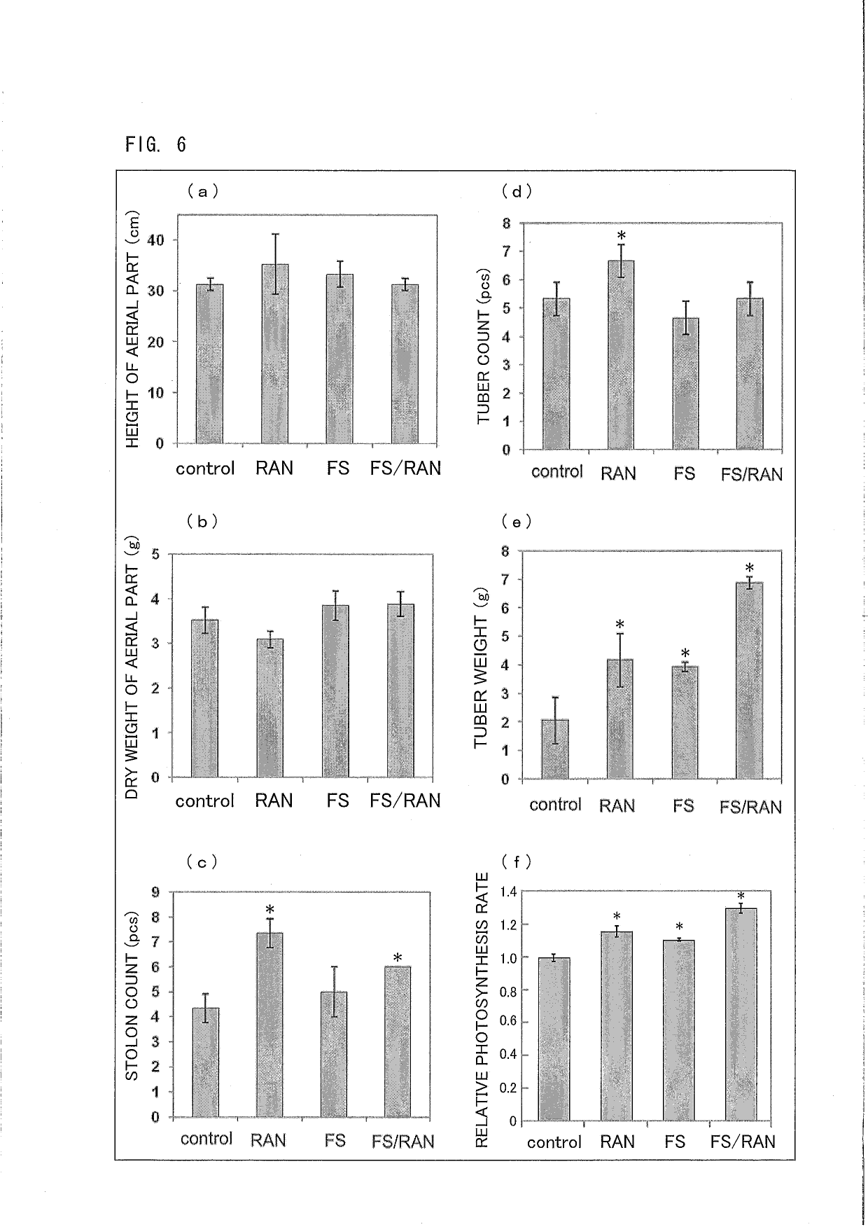

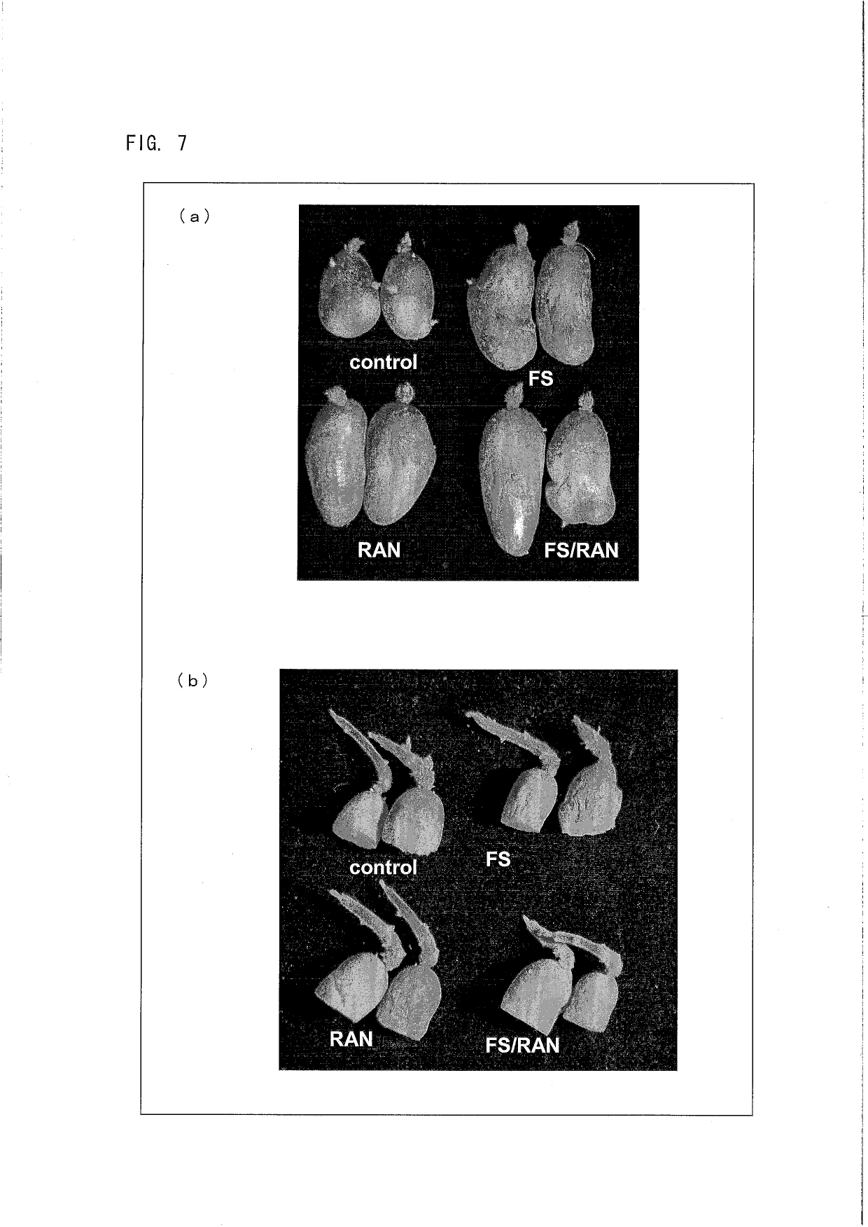

After five weeks of growing, all genomic DNAs were extracted from a leaf of each of the transformants, and whether or not NPTII, the RAN gene, and the FBPase/SBPase gene had been introduced was checked by PCR (FIG. 3). Whether or not the RAN protein and the FBPase/SBPase protein were expressed was also checked by western blotting (FIG. 4).

The RAN transgenic potato plant, in which introduction and expression of the RAN gene were confirmed, and the FS/RAN transgenic potato plant, in which introduction and expression of the RAN gene and introduction and expression of the FBPase/SBPase gene were confirmed, were measured in terms of height and dry weight of an aerial part, number of stolons, number and weight of tubers, and photosynthesis rate (photosynthesis CO₂ fixation rate) (FIG. 6).

Note that photosynthesis rates (photosynthesis CO₂ fixation rate) were measured by use of a portable photosynthesis and transpiration measurement system LI-6400 (LI-COR) at a light intensity of 1000 μmol photons/m²/sec and a CO₂ concentration of 350 ppm.

[4. Results]

FIG. 3 shows agarose gel electrophoretograms in which whether or not genes were introduced into the transgenic potato plants, which had been obtained in the examples and the comparative example, were checked by PCR. In FIG. 3, the lanes indicated as “RAN” are results of the potato plant (example 1) into which only the Ran gene was introduced, the lanes indicated as “FS” are results of the potato plant (comparative example) into which only the FBPase/SBPase gene was introduced, and the lanes indicated as “FS/RAN” are results of the potato plant (example 2) into which the Ran gene and the FBPase/SBPase gene were introduced. Further, in FIG. 3, “M” shows molecular-weight markers, “+” shows positive controls, “Wt” shows results of a corresponding wild strain, and “CV” shows results of the potato plant into which only a vector, which was a control, was introduced. In FIG. 3, “NPTII” shows whether or not DNA derived from a vector was detected, “CLRan1” shows whether or not the Ran gene was detected, and “FBP/SBP” shows whether or not the FBPase/SBPase gene was detected.

From the “RAN” lanes in FIG. 3, only the Ran gene and DNA derived from the vector were detected. From the “FS” lanes in FIG. 3, only the FBPase/SBPase gene and DNA derived from the vector were detected. From the “FS/RAN” lanes, the Ran gene, the FBPase/SBPase gene, and DNA derived from the vector were detected. Accordingly, it was confirmed that transgenic potato plants, into which respective desired genes had been introduced, were obtained.

FIG. 4 shows results of checking, by western blotting, whether or not FBPase/SBPase and the RAN protein were expressed in each of the transgenic potato plants (fifth week of cultivation) obtained in the examples and the comparative example. (a) of FIG. 4 shows whether or not the Ran protein was detected, and (b) of FIG. 4 shows whether or not FBPase/SBPase was detected. In FIG. 4, the lanes indicated as “RAN” are results of the potato plant (example 1) into which only the Ran gene was introduced, the lanes indicated as “FS” are results of the potato plant (comparative example) into which only the FBPase/SBPase gene was introduced, and the lanes “FS/RAN” are results of the potato plant (example 2) into which the Ran gene and the FBPase/SBPase gene were introduced. In each of the transgenic potato plants, it was confirmed that the Ran gene and/or the FBPase/SBPase gene were expressed and the proteins encoded by the respective genes were expressed (excluding the lanes 1 and 3 of “FS/RAN” in (b) of FIG. 4). In view of the results of western analysis, it was decided that transformants used in the lane 1 of RAN, the lane 1 of FS, and the lane 2 of FS/RAN in the western analysis were to be used in the following analysis.

FIG. 5 shows a photograph ((a) of FIG. 5) of entire plant bodies of the transgenic potato plants (fifth week of cultivation) obtained in the examples and the comparative example, a photograph ((b) of FIG. 5) of portions around rhizome parts and root parts of the transgenic potato plants (fifth week of cultivation), and a photograph ((c) of FIG. 5) of tubers harvested from the transgenic potato plants (fifth week of cultivation). In FIG. 5, the potato plant indicated as “RAN” is a result of the potato plant (example 1) into which only the Ran gene was introduced, the potato plant indicated as “FS” is a result of the potato plant (comparative example) into which only the FBPase/SBPase gene was introduced, the potato plant indicated as “FS/RAN” is a result of the potato plant (example 2) into which the Ran gene and the FBPase/SBPase gene were introduced, and the potato plant indicated as “control” is a result of the potato plant into which only the control vector was introduced. Note that the inventors of the present invention have confirmed (not shown) that a potato strain (control) into which only the control vector was introduced and a corresponding wild strain are not different from each other in terms of: height and dry weight of an aerial part; number of stolons; size, number, and weight of tubers; and photosynthesis rate (photosynthesis CO₂ fixation rate). Accordingly, comparing the examples or the comparative example with the control can be regarded as an equivalent of comparing the examples or the comparative example with the corresponding wild strain.

The results shown in FIG. 5 indicate that each of the potato plant (example 1) into which only the Ran gene was introduced, the potato plant (comparative example) into which only the FBPase/SBPase gene was introduced, and the potato plant (example 2) into which the Ran gene and the FBPase/SBPase gene were introduced clearly exhibited an increase in size of tubers as compared the control (see (c) of FIG. 5 in particular). Further, unexpectedly, the potato plant (example 2) into which the Ran gene and the FBPase/SBPase gene had been introduced had no change in height of the plant body itself (height of the aerial part) and only exhibited the change in size of tubers (see (a) and (c) of FIG. 5 in particular).

FIG. 6 shows results of measuring the height and dry weight of an aerial part, the number of stolons, the number and weight of tubers, and the photosynthesis rate (photosynthesis CO₂ fixation rate) of each of the transgenic potato plants (fifth week of cultivation) obtained in the examples and the comparative example. (a) of FIG. 6 is a graph of comparison between the transgenic potato plants (fifth week of cultivation) in terms of height of an aerial part, (b) of FIG. 6 is a graph of comparison between the transgenic potato plants (fifth week of cultivation) in terms of dry weight of an aerial part, (c) of FIG. 6 is a graph of comparison between the transgenic potato plants (fifth week of cultivation) in terms of number of stolons (stolon count), (d) of FIG. 6 is a graph of comparison between the transgenic potato plants (fifth week of cultivation) in terms of number of tubers (tuber count), (e) of FIG. 6 is a graph of comparison between the transgenic potato plants (fifth week of cultivation) in terms of weight of tubers (tuber weight), (f) of FIG. 6 is a graph of comparison between the transgenic potato plants (fifth week of cultivation) in terms of photosynthesis rate. Note that “relative photosynthesis rate” in (f) of FIG. 6 indicates a relative value obtained in a case where a photosynthesis rate of the control is 1.0. The data of FIG. 6 was subjected to a significant difference test in accordance with a T-test, and “*” assigned to a result indicates that the result had a significant difference as compared with the control (significance level: 5% or less).

As the result shown in (a) of FIG. 6 indicates, the plant body (example 1) into which the RAN gene had been introduced and the plant body (example 2) into which the RAN gene and the FBPase/SBPase gene had been introduced did not exhibit a significant difference in height of an aerial part as compared with control. As the result shown in (b) of FIG. 6 indicates, the plant body (example 1) into which the RAN gene had been introduced and the plant body (example 2) into which the RAN gene and the FBPase/SBPase gene had been introduced did not exhibit a significant difference in dry weight of an aerial part as compared with the control. However, the plant body (example 1) into which the Ran gene had been introduced exhibited a significant increase in number of stolons as compared with the control, and the plant body (example 2) into which the Ran gene and the FBPase/SBPase gene had been introduced also exhibited a significant increase in number of stolons as compared with the control ((c) of FIG. 6). In terms of the number of tubers per individual plant, the plant body (example 1) into which the Ran gene had been introduced exhibited a significant increase as compared with the control, but the plant body (example 2) into which the Ran gene and the FBPase/SBPase gene had been introduced did not exhibit a significant difference ((d) of FIG. 6). A weight of tubers per individual plant increased by about 2 times in the plant body (example 1) into which the Ran gene had been introduced, and increased by about 3.5 times in the plant body (example 2) into which the Ran gene and the FBPase/SBPase gene had been introduced ((e) of FIG. 6). A photosynthesis rate of the plant body (example 1) into which the Ran gene had been introduced increased significantly by about 1.2 times as compared with the control, and a photosynthesis rate of the plant body (example 2) into which the Ran gene and the FBPase/SBPase gene had been introduced increased significantly by about 1.5 times as compared with the control ((f) of FIG. 6).

The following finding was made from the results shown in FIG. 6. That is, introduction of a Ran gene increased the number of stolons (i.e., accelerates development of the stolons). Since the number of parts in which starch was accumulated was thus increased, synthesis of starch was accelerated (i.e., a photosynthesis rate was increased). Consequently, the number and weight of tubers increased as compared with the control (i.e., tuber production ability was improved).

It was also found out that in a case where the FBPase/SBPase gene was introduced, development of stolons was not particularly affected but a photosynthesis rate was increased, so that the number and weight of tubers increased as compared with the control (i.e., tuber production ability was improved). It was also found out that in a case where the Ran gene and the FBPase/SBPase gene were introduced, (i) the number of stolons was increased (i.e., development of stolons was accelerated) and, since the number of parts in which starch was accumulated was thus increased, synthesis of starch was accelerated (i.e., a photosynthesis rate was increased) and (ii) the introduction of the FBPase/SBPase gene increased a photosynthesis rate, so that the number and weight of tubers increased further notably as compared with the control (i.e., tuber production ability was improved).

Next, germination abilities of tubers of the transgenic potato plants obtained in the examples and the comparative example were analyzed (germination ability test). A tuber of each of the transgenic potato plants which had been preserved at 4° C. for one month was left still at 25° C. in a dark place in a state where the tuber was placed on a sheet of filter paper impregnated with water and was covered with a resin film so as to be prevented from drying.

Results of this are shown in FIG. 7. (a) and (b) of FIG. 7 are photographs of the tubers of the transgenic potato plants obtained in the examples and the comparative example, the photograph of (a) being taken one month after the tubers were subjected to the germination ability test, the photograph of (b) being taken after two month after the tubers were subjected to the germination ability test. In FIG. 7, the potato plants indicated as “RAN” are results of the potato plant (example 1) into which only the Ran gene was introduced, the potato plants indicated as “FS” are results of the potato plant (comparative example) into which only the FBPase/SBPase gene was introduced, the potato plants indicated as “FS/RAN” are results of the potato plant (Example 2) into which the Ran gene and the FBPase/SBPase gene were introduced, the potato plants indicated as “control” are results of the potato plant into which only the control vector was introduced.

As is indicated by FIG. 7, all of the transgenic potato plants germinate to the same extent. This result indicates that the tubers of all of the transgenic potato plants had germination ability. It was thus found that also the potato plant into which the Ran gene and the FBPase/SBPase gene were introduced maintained its germination ability and was capable of leaving a next generation and even serving as a seed potato in the same manner as the control.

Note that the method for producing a stolon-forming plant in accordance with the present invention can include a step of carrying out the above-described germination ability test in order to check whether or not a transformed plant is capable of forming a next generation plant.

### INDUSTRIAL APPLICABILITY

The present invention makes it possible to provide a method for producing a stolon-forming plant having an improved tuber production ability as compared with a corresponding wild strain, and a stolon-forming plant produced by the method. The present invention also makes it possible to produce a method for producing a stolon-forming plant having an improved stolon formation ability as compared with a corresponding wild strain, and a stolon-forming plant produced by the method. Therefore, the present invention can be expected to contribute to the field of agriculture, food industry, and the like.

1. A method for producing a stolon-forming plant having an improved tuber production ability as compared with a corresponding wild strain, the method comprising the step of:
introducing, into a stolon-forming plant, a Ran gene derived from Citrullus lanatus.

2. The method as set forth in claim 1, wherein the Ran gene is the following (A) or (B):
(A) a polynucleotide consisting of the base sequence of SEQ ID NO: 1 or 2; and
(B) a polynucleotide that is hybridizable, under a stringent condition, with a polynucleotide consisting of a complementary base sequence of the polynucleotide consisting of the base sequence of SEQ ID NO: 1 or 2 and encodes a polypeptide having an activity of improving a tuber production ability.

3. The method as set forth in claim 1, wherein the Ran gene is a polynucleotide encoding a polypeptide of the following (C) or (D):
(C) a polypeptide consisting of the amino acid sequence of SEQ ID NO: 3 or 4; and
(D) a polypeptide (i) consisting of an amino acid sequence in which one or several amino acids are substituted, deleted, or added in the polypeptide consisting of the amino acid sequence of SEQ ID NO: 3 or 4 and (ii) having an activity of improving a tuber production ability.

4. A method as set forth in claim 1, further comprising the step of:
introducing, into the stolon-forming plant, a polynucleotide encoding fructose-1,6-bisphosphatase/sedoheptulose-1,7-bisphosphatase derived from a blue-green alga.

5. The method as set forth in claim 4, wherein the polynucleotide encoding fructose-1,6-bisphosphatase/sedoheptulose-1,7-bisphosphatase derived from a blue-green alga is the following (E) or (F):
(E) a polynucleotide consisting of the base sequence of SEQ ID NO: 5; and
(F) a polynucleotide that is hybridizable, under a stringent condition, with a polynucleotide consisting of a complementary base sequence of the polynucleotide consisting of the base sequence of SEQ ID NO: 5 and encodes a polypeptide having fructose-1,6-bisphosphatase/sedoheptulose-1,7-bisphosphatase activity.

6. The method as set forth in claim 4, wherein the polynucleotide encoding fructose-1,6-bisphosphatase/sedoheptulose-1,7-bisphosphatase derived from a blue-green alga is a polynucleotide encoding a polypeptide of the following (G) or (H):
(G) a polypeptide consisting of the amino acid sequence of SEQ ID NO: 6; and
(H) a polypeptide (i) consisting of an amino acid sequence in which one or several amino acids are substituted, deleted, or added in the polypeptide consisting of the amino acid sequence of SEQ ID NO: 6 and (ii) having fructose-1,6-bisphosphatase/sedoheptulose-1,7-bisphosphatase activity.

7. A stolon-forming plant having an improved tuber production ability as compared with a corresponding wild strain, the stolon-forming plant being produced by a method recited in claim 1.

8. A kit for improving a tuber production ability of a stolon-forming plant, the kit comprising:
a Ran gene derived from Citrullus lanatus; and
a polynucleotide encoding fructose-1,6-bisphosphatase/sedoheptulose-1,7-bisphosphatase derived from a blue-green alga.

9. A method for producing a stolon-forming plant having an improved stolon formation ability as compared with a corresponding wild strain, the method comprising the step of:
introducing, into a stolon-forming plant, a Ran gene derived from Citrullus lanatus.

10. The method as set forth in claim 9, wherein the Ran gene is the following (A) or (B):
(A) a polynucleotide consisting of the base sequence of SEQ ID NO: 1 or 2; and
(B) a polynucleotide that is hybridizable, under a stringent condition, with a polynucleotide consisting of a complementary base sequence of the polynucleotide consisting of the base sequence of SEQ ID NO: 1 or 2 and encodes a polypeptide having an activity of improving a stolon formation ability.

11. The method as set forth in claim 9, wherein the Ran gene is a polynucleotide encoding a polypeptide of the following (C) or (D):
(C) a polypeptide consisting of the amino acid sequence of SEQ ID NO: 3 or 4; and
(D) a polypeptide (i) consisting of an amino acid sequence in which one or several amino acids are substituted, deleted, or added in the polypeptide consisting of the amino acid sequence of SEQ ID NO: 3 or 4 and (ii) having an activity of improving a stolon formation ability.

12. A stolon-forming plant having an improved stolon formation ability as compared with a corresponding wild strain, the stolon-forming plant being produced by a method recited in claim 9.

13. A kit for improving a stolon formation ability of a stolon-forming plant, the kit comprising a Ran gene derived from Citrullus lanatus.


SEQUENCE LISTING (10 sequences)

SEQ ID NO: 1 - DNA, 666 bp, Citrullus lanatus
atggctttgccggaccagaagactgttgattatccgagtttcaagcttgttattgttggtgatggtggcactggaaaaacaacatttgtgaaaagacatctcacaggggagttcgaaaagaaatacgaaccaaccattggtgtggaggtgcacccattggacttcttcactaactgtggaaaaattagattttactgttgggacactgctgggcaggagaaatttggtggtttacgtgatggctactacatccatgggcaatgtgcaatcatcatgtttgatgttactgccagattgacatataaaaatgttccgacgtggcaccgtgatctttgcagagtgtgtgagaatattccaatagttctttgtggaaacaaggtcgatgtgaaaaacaggcaggttaaggccaagcaagtcacattccacaggaagaagaacctacagtactatgaaatatctgcaaagagtaactacaacttcgaaaaaccattcctgtacttggccagaaagttggctggggatccaaacattcatttcgtggagtctccagctctggctcctcctgaagtacacattgatttggctgcccagcaacagcatgaagctgaattagcggcagcagcaagtcaacctcttcccgacgatgacgacgacgcgttcgagtaa

SEQ ID NO: 2 - DNA, 666 bp, Citrullus lanatus
atggcgctaccaaatcaggccaccgtagattaccctagcttcaagcttgtaattgtcggagatggtggaacagggaagacaacctttgttaaacgtcatcttactggagagttcgagaagaaatatgaaccgacaatcggagttgaagttcatccactggacttctttacaaactatggaaagataaggttttactgctgggatactgctggacaggagaagtttggtggacttagagatggttactatattcatgggcaatgtgccattatcatgtttgatgtcactgctcgtttaacatacaaaaatgtcccaacatggcatcgtgatctctgcagggtttgtgagaacatacccatcgtattgtgtggaaacaaggtcgacgtgaaaaacagacaggttaaagcaaagcaggtcaccttccacaggaagaaaaatcttcagtattacgaaatctctgcaaagagtaattacaactttgagaagcccttcttgtaccttgccagaaagcttgctggtaatccggagcttcacttcgttgaatcacctgccctggctcctcctgaagttcaaattgacttggccgcacaagcacagcatgaagcagagttggcgcaagctgctgcacaacctcttcctgatgaagatgacgatgcatttgagtaa

SEQ ID NO: 3 - protein, 221 aa, Citrullus lanatus
MALPDQKTVDYPSFKLVIVGDGGTGKTTFVKRHLTGEFEKKYEPTIGVEVHPLDFFTNCGKIRFYCWDTAGQEKFGGLRDGYYIHGQCAIIMFDVTARLTYKNVPTWHRDLCRVCENIPIVLCGNKVDVKNRQVKAKQVTFHRKKNLQYYEISAKSNYNFEKPFLYLARKLAGDPNIHFVESPALAPPEVHIDLAAQQQHEAELAAAASQPLPDDDDDAFE

SEQ ID NO: 4 - protein, 221 aa, Citrullus lanatus
MALPNQATVDYPSFKLVIVGDGGTGKTTFVKRHLTGEFEKKYEPTIGVEVHPLDFFTNYGKIRFYCWDTAGQEKFGGLRDGYYIHGQCAIIMFDVTARLTYKNVPTWHRDLCRVCENIPIVLCGNKVDVKNRQVKAKQVTFHRKKNLQYYEISAKSNYNFEKPFLYLARKLAGNPELHFVESPALAPPEVQIDLAAQAQHEAELAQAAAQPLPDEDDDAFE

SEQ ID NO: 5 - DNA, 1350 bp, Synechococcus
  misc_feature (69)..(70): n is a, c, g, or t
cgtcgcccgctccatgcccgcagctgcgcctttgatgccgcggaagatattgccgccaactaacgatannagtcactgcgatcgcaactaaagccagagatgtgaggaggggatccggcctttggtagactcaactgttggaatccccagaagcaatcatccgtaaggagtcaggacggcgtggagaagacgatcggtctcgagattattgaagttgtcgagcaggcagcgatcgcctcggcccgcctgatgggcaaaggcgaaaagaatgaagccgatcgcgtcgcagtagaagcgatgcgggtgcggatgaaccaagtggaaatgctgggccgcatcgtcatcggtgaaggcgagcgcgacgaagcaccgatgctctatatcggtgaagaagtgggcatctaccgcgatgcagacaagcgggctggcgtaccggctggcaagctggtggaaatcgacatcgccgttgacccctgcgaaggcaccaacctctgcgcctacggtcagcccggctcgatggcagttttggccatctccgagaaaggcggcctgtttgcagctcccgacttctacatgaagaaactggctgcacccccagctgccaaaggcaaagagacatcaataaagtccgcgaccgaaaacctgaaaattctctcggaatgtctcgatcgcgccatcgatgaattggtggtcgtggtcatggatcgtccccgccacaaagagctaatccaagagatccgccaagcgggtgcccgcgtccgtctgatcagcgatggtgacgtttcggccgcgatctcctgcggttttgctggcaccaacacccacgccctgatgggcatcggtgcagctcccgagggtgtgatttcggcagcagcaatgcgttgcctcggcgggcacttccaaggccagctgatctacgacccagaagtggtcaaaaccggcctgatcggtgaaagccgtgagagcaacatcgctcgcctgcaagaaatgggcatcaccgatcccgatcgtgtctacgacgcgaacgaactggcttcgggtcaagaagtgctgtttgcggcttgcggtatcaccccgggcttgctgatggaaggcgtgcgcttcttcaaaggcggcgctcgcacccagagcttggtgatctccagccagtcacggacggctcgcttcgttgacaccgttcacatgttcgacgatgtcaaaacggttagcctgccgttaattcctgatcccaaatggcggccggagcggtagaacgggtatagctcgatcgcttcggtcgttgtttttcagcgaatccatttgcgatcgcttttcaaacccttttttcgtcaaccttctttaaacggcctc

SEQ ID NO: 6 - protein, 356 aa, Synechococcus
MEKTIGLEIIEVVEQAAIASARLMGKGEKNEADRVAVEAMRVRMNQVEMLGRIVIGEGERDEAPMLYIGEEVGIYRDADKRAGVPAGKLVEIDIAVDPCEGTNLCAYGQPGSMAVLAISEKGGLFAAPDFYMKKLAAPPAAKGKETSIKSATENLKILSECLDRAIDELVVVVMDRPRHKELIQEIRQAGARVRLISDGDVSAAISCGFAGTNTHALMGIGAAPEGVISAAAMRCLGGHFQGQLIYDPEVVKTGLIGESRESNIARLQEMGITDPDRVYDANELASGQEVLFAACGITPGLLMEGVRFFKGGARTQSLVISSQSRTARFVDTVHMFDDVKTVSLPLIPDPKWRPER

SEQ ID NO: 7 - DNA, 28 bp, Artificial Sequence
  Description of Artificial Sequenceartificially synthesized primer sequence
aaaaagcaggctccttttccaatggctt

SEQ ID NO: 8 - DNA, 28 bp, Artificial Sequence
  Description of Artificial Sequenceartificially synthesized primer sequence
agaaagctgggttttttactcgaacgcg

SEQ ID NO: 9 - DNA, 20 bp, Artificial Sequence
  Description of Artificial Sequenceartificially synthesized primer sequence
gaattcgcatgcctgcaggt

SEQ ID NO: 10 - DNA, 20 bp, Artificial Sequence
  Description of Artificial Sequenceartificially synthesized primer sequence
gaattcccgatctagtaaca